(19) United States
(12) Patent Application Publication
(10) Pub. No.: US 2020/0308521 A1
(43) Pub. Date: Oct. 1, 2020

(54) CULTURE APPARATUS

(71) Applicant: TAKASAGO ELECTRIC, INC., Nagoya-shi (JP)

(72) Inventors: Naoya ASAI, Nagoya-shi (JP); Daiki KAWATA, Nagoya-shi (JP); Hiroyuki SUGIURA, Nagoya-shi (JP); Yusuke OGIHARA, Nagoya-shi (JP)

(73) Assignee: TAKASAGO ELECTRIC, INC., Nagoya-shi (JP)

(21) Appl. No.: 16/829,521

(22) Filed: Mar. 25, 2020

(30) Foreign Application Priority Data
Mar. 25, 2019 (JP) 2019-057224

(51) Int. Cl. C12M 3/00; C12M 1/00; C12M 1/34

(52) U.S. Cl. CPC C12M 23/44; C12M 41/00; C12M 29/20; C12M 29/04

(57) ABSTRACT

A culture apparatus which supplies a culture medium, which is a cell culture solution, to culture cells includes a culture cartridge including a culture space where the cells can be seeded, a supply path for supplying the culture medium to the culture space, and a collection path for collecting the culture medium passing through the culture space. The culture cartridge is configured to be attachable and detachable, and includes connecting parts and for connecting, when attached, the culture space to the supply path and the collection path.

### BACKGROUND OF THE INVENTION

### Field of the Invention

The present invention relates to a culture apparatus for use in cell culture.

### Description of the Related Art

Conventionally, studies toward practical use of iPS cells and ES cells have been promoted. One purpose of these studies is to achieve regenerative medicine in which cells are cultured and their culture is transplanted to humans. One key point to determine the success or failure of this regenerative medicine is reliability and accuracy of culture of cells and so forth. During culturing by seeding cells in a culture vessel filled with a culture medium, which is a culture solution, the culture medium is appropriately replaced, thereby allowing an improvement in accuracy of culture.

For example, in Japanese Patent Laid-Open No. 2017-79633, a culture apparatus which automatically replaces a culture medium in a culture vessel is suggested. In this culture apparatus, the culture medium can be replaced by supplying a fresh culture medium while discharging the culture medium in the culture vessel by a pump in an airtight structure. This culture apparatus is an apparatus with excellent characteristics in which the culture medium in the culture vessel can be automatically replaced and the work load of replacing the culture medium can be reduced.

### SUMMARY OF THE INVENTION

However, the above-described conventional culture apparatus has the following problem. That is, while trouble with work can be reduced because the culture medium can be automatically replaced, there is a problem in which it takes trouble to perform preparation work, such as accommodating cells in the culture vessel.

The present invention was made in view of the above-described conventional problem, and is to provide a culture apparatus capable of reducing trouble with work.

The present invention resides in a culture apparatus which supplies a culture medium, which is a cell culture solution, to culture cells, including

a culture cartridge including a culture space where the cells can be seeded,

a supply path for supplying the culture medium to the culture space, and

a collection path for collecting the culture medium passing through the culture space, wherein

the culture cartridge is attachable and detachable, and includes a connecting part for connecting, when attached, the culture space to the supply path and the collection path.

### Advantageous Effects of the Invention

The culture apparatus of the present invention is an apparatus which cultures cells seeded in the culture space of the culture cartridge which is attachable and detachable. In this culture apparatus, when the culture cartridge is attached, the supply path and the collection path of the culture medium can be connected to the culture space, thereby allowing cell culture. In the culture apparatus of the present invention, for culture preparation work, cells can be seeded by handling the culture cartridge not the main body of the culture apparatus. The culture cartridge is small compared with the main body provided with the supply path and the collection path of the culture medium. The preparation work for seeding cells can be performed relatively easily with the relatively small-sized culture cartridge. And, by attaching the culture cartridge where cells are seeded to the main body, the preparation work for cell culture can be nearly completed.

Furthermore, in the culture apparatus of the present invention, the culture cartridge can be removed from the main body, for example, after cell culturing ends. The cell culture state can be maintained as it is in the culture space of the culture cartridge even if the culture cartridge is removed from the main body. For example, for performing work for postprocessing, it is possible to carry the culture cartridge to a place where work can be easily performed to perform work for postprocessing.

As described above, the culture apparatus of the present invention is an apparatus with excellent characteristics capable of efficiently performing work and reducing trouble with work.

### BRIEF DESCRIPTION OF THE DRAWINGS

FIG. 1 is a diagram depicting a culture apparatus;

FIG. 2 is a diagram depicting an apparatus main body;

FIG. 3 is a block diagram depicting an electrical configuration of the culture apparatus;

FIG. 4 is a descriptive diagram depicting a holding structure of culture cartridges;

FIG. 5 is a diagram depicting a cartridge holder;

FIG. 6 is a descriptive diagram of the structure of the culture cartridge;

FIG. 7 is a perspective view of a cell culture cartridge viewed from an upstream side;

FIG. 8 is a perspective view of the cell culture cartridge viewed from a downstream side;

FIG. 9 is a cross-sectional view depicting an inner structure of the culture cartridge; and

FIG. 10 is a descriptive diagram depicting paths of air and a culture medium in the culture apparatus.

### DETAILED DESCRIPTION OF THE PREFERRED EMBODIMENTS

### Embodiments

Embodiments of the present invention are specifically described by using the following embodiments:

### First Embodiment

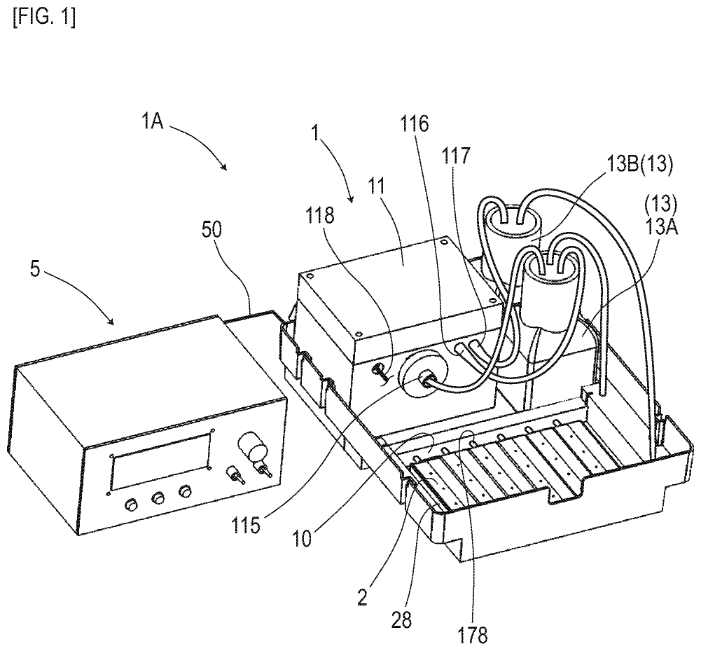

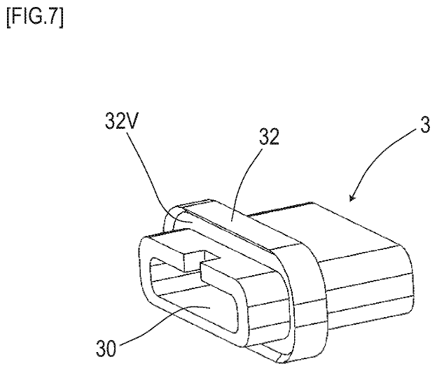

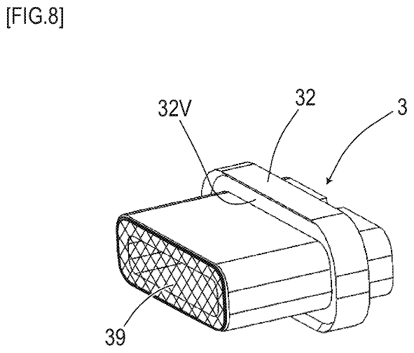

The present embodiment is an example regarding a cartridge-type culture apparatus 1A. Details of this are described by using FIG. 1 to FIG. 10.

The culture apparatus 1A (FIG. 1) is an apparatus which supplies a culture medium, which is a culture solution to cells for cell culture. This culture apparatus 1A is configured to include an apparatus main body (main body of the culture apparatus) 1 to which culture cartridges 2 are attachable and a controller unit 5 which controls the apparatus main body 1. For example, the apparatus main body 1 accommodated in an incubator not depicted, which is an apparatus for keeping the environment constant, is controlled by the external controller unit 5 electrically connected via a control cable 50.

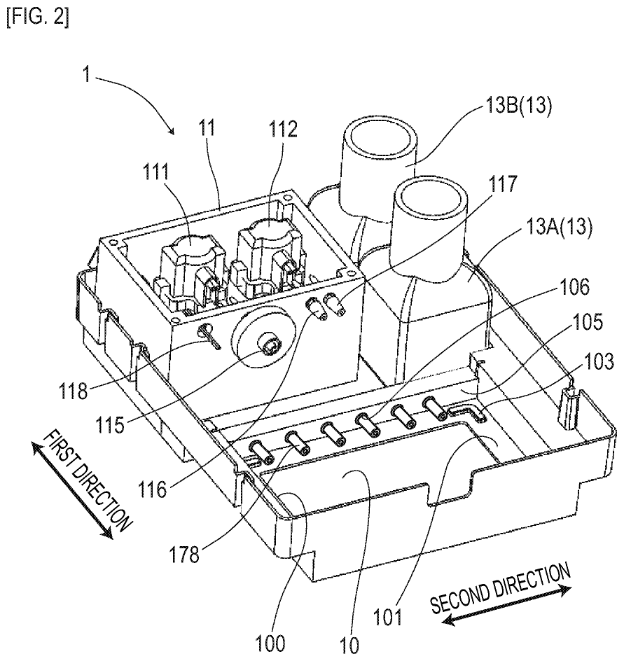

The apparatus main body 1 (FIG. 1 and FIG. 2) includes a box-shaped pump box 11 having pumps 111, 112 and so forth for circulating the culture medium accommodated therein, a bottle 13 which accommodates the culture medium, an accommodating part 10 as a space for accommodating the culture cartridges 2 and so forth. In the following description, a longer direction on an upper surface of the apparatus main body 1 having a rectangular shape is taken as the first direction, and a shorter direction is taken as the second direction.

The box-shaped pump box 11 (FIG. 2) is arranged so as to be close to a corner part at one location on an upper surface of the apparatus main body 1 having a rectangular shape, and is disposed so as to have margins in the first direction and the second direction. In a margin in the first direction adjacent to the pump box 11, the above-described accommodating part 10 is arranged. In a margin in the second direction adjacent to the pump box 11, two bottles 13 are adjacently arranged along a depth direction. In FIG. 2, for depiction of the pumps 111 and 112 and so forth, depiction of a lid of the pump box 11 is omitted. Also in the drawing, depiction of tubes forming paths for air and a culture medium and so forth is omitted.

One of the two bottles 13 is a supply bottle 13A for supplying the culture medium to the culture cartridges 2. The other is a collection bottle 13B for collecting the culture medium discharged from the culture cartridges 2. The supply bottle 13A and the collection bottle 13B are arranged, as the pump box 11, on one side in the first direction. By this, a wide space for providing the accommodating part 10 is allocated on the opposite side in the first direction.

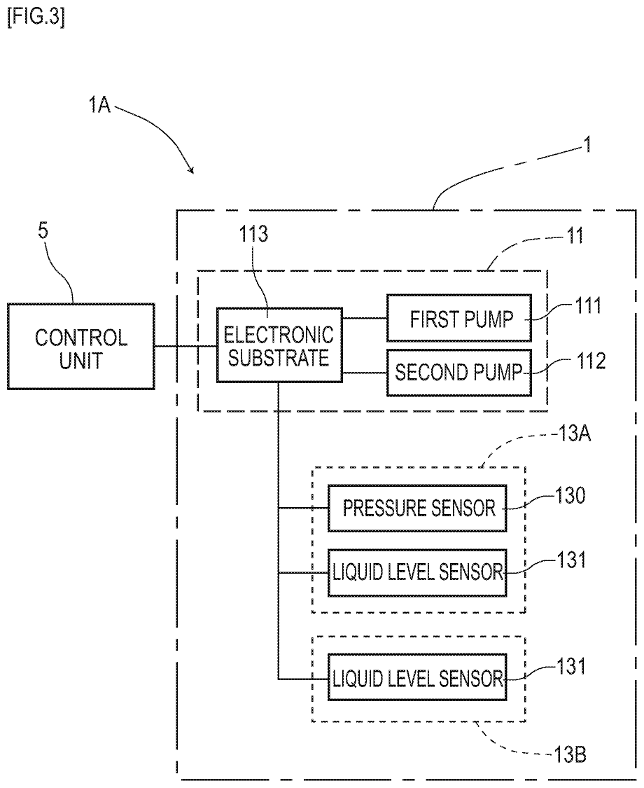

The pump box 11 (FIG. 2) accommodates therein the first pump 111, the second pump 112, an electronic substrate 113 (FIG. 3) and so forth. The first pump 111 is a pump which pumps air (atmosphere) suctioned from outside to the supply bottle 13A. The second pump 112 is a pump for recirculating the culture medium in the collection bottle 13B to the supply bottle 13A. In addition to the first pump 111 and the second pump 112, the third pump for collecting the culture medium passing through the culture cartridges 2 into the collection bottle 13B may be provided.

The electronic substrate 113 is the substrate for exchanging various signal with the control unit 5 (refer to FIG. 3). This electronic substrate 113 controls the first pump 111, the second pump 112 and so forth based on control signals from the control unit 5. Also, the electronic substrate 113 processes sensor signals from a pressure sensor 130 (FIG. 3) and a liquid level sensor 131 disposed to the supply bottle 13A, a liquid level sensor 131 disposed to the collection bottle 13B and so forth, and also inputs measurement signals based on the sensor signals to the control unit 5.

On the outer peripheral surface of the box-shaped pump box 11, as in FIG. 1 and FIG. 2, an air discharge port 115, a suction port 117 which suctions the culture medium in the collection bottle 13B, a discharge port 116 which discharges the culture medium toward the supply bottle 13A and so forth are provided in a standing manner, and signal lines 118 for various sensor signals and so forth are penetratingly arranged. In FIG. 2, depiction of a silicone tube connected to each port is omitted. Also in FIG. 1 and FIG. 2, wires of the signal lines 118 are broken partway to omit depiction of a wiring mode.

The accommodating part 10 is dug down from an upper plane to form a recessed shape, and has an opening shape toward the upper plane, the opening shape being a rectangular shape which is elongated in the second direction. The opening of the accommodating part 10 is one example of an observation allowing part for allowing observation of a cell culture state. The bottom surface of the accommodating part 10 is a holed bottom surface where a through window 100 is formed. The through window 100 is a window in the one size smaller rectangular shape than the opening shape of the recessed accommodating part 10. This through window 100 forms one example of the observation allowing part for allowing observation of the cell culture state. A shelf-shaped bottom surface 101 is formed between the inner side surface of the accommodating part 10 and the through window 100. The shelf-shaped bottom surface 101 is a support surface of a cartridge holder 28 which holds the culture cartridges 2.

At each corner part of the bottom surface 101 of the accommodating part 10, as in FIG. 2, an L-shaped ridge part 103 for positioning a corner part of the frame-shaped cartridge holder 28 is provided. Also, of the inner side surfaces of the accommodating part 10, an inner side surface 105 facing the first direction has tube holes 106 bored at six locations for penetratingly arranging silicone tubes 178. The silicone tubes 178 penetrating through the respective tube holes 106 are tubes for connecting to the culture cartridges 2. Each silicone tube 178 has a margin in length to some extent so as to be drawn out when connected to the culture cartridge 2 for allowing connection work to be performed.

The six silicone tubes 178 penetratingly arranged on the inner side surface 105 on the pump box 11 side form supply paths for supplying the culture medium in the supply bottle 13A to the culture cartridges 2. The six silicone tubes 178 penetratingly arranged on the inner side surface 105 on the opposite side form collection paths for collecting the culture medium discharged from the culture cartridges 2 to the collection bottle 13B.

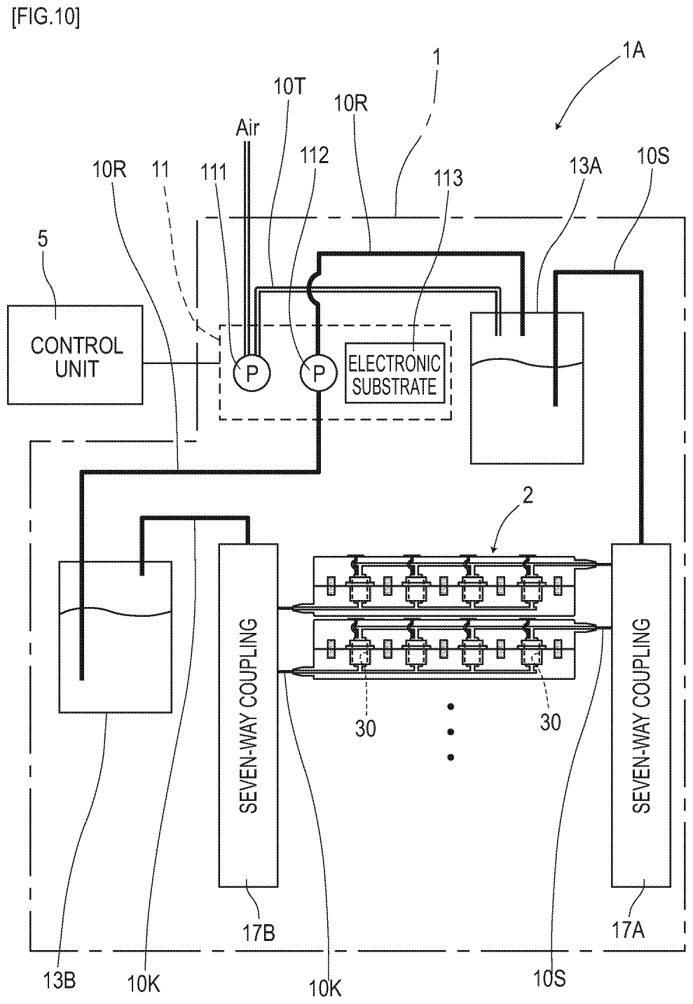

The six silicone tubes 178 forming supply paths are connected to a seven-way coupling 17A (FIG. 10) on an upstream side provided inside the apparatus main body 1. Also, the six silicone tubes 178 forming collection paths are connected to a seven-way coupling 17B (FIG. 10) on a downstream side provided inside the apparatus main body 1. To the seven-way coupling 17A on the upstream side, in addition to the six silicone tubes 178 forming supply paths, a silicone tube 178 forming a supply path provided to extend from the supply bottle 13A is connected. To the seven-way coupling 17B on the downstream side, in addition to the six silicone tubes 178 forming collection paths, a silicone tube 178 forming a collection path provided to extend from the collection bottle 13B is connected. In the present example, a resin-molded product is adopted as a housing of the apparatus main body 1, and a coupling structure forming the seven-way couplings 17A and 17B is simultaneously molded at the time of the resin molding of the housing.

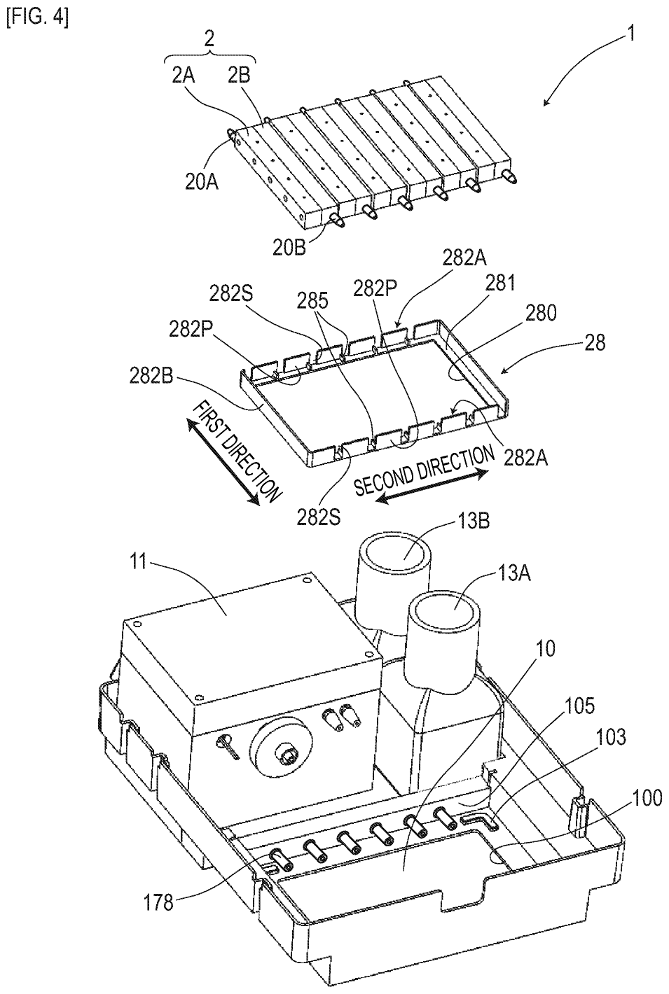

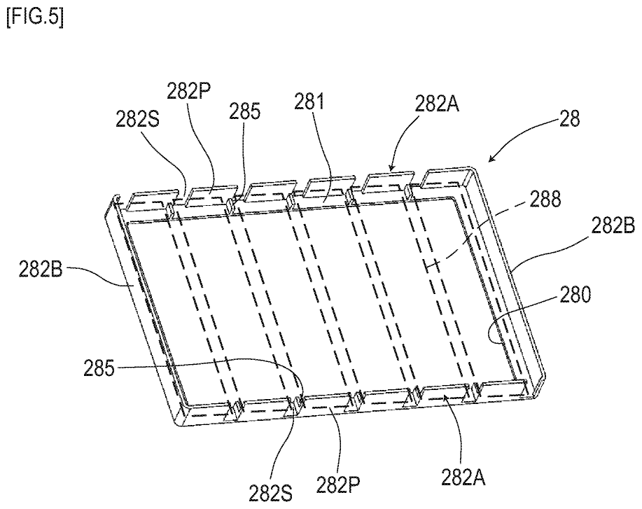

The cartridge holder 28 (FIG. 4 and FIG. 5) is a holder for holding six culture cartridges 2 in parallel. The cartridge holder 28 has a frame shape by outer peripheral side walls 282A and 282B provided along the four sides of a rectangle and an annular bottom part 281 forming a base of the outer peripheral side walls 282A and 282B. On an inner peripheral side of the annular bottom part 281, a through window 280 having the same shape as that of the through window 100 of the accommodating part 10 is formed. An upper opening of the frame-shaped cartridge holder 28 and the through window 280 form one example of the observation allowing part which allows observation of the cell culture state.

The cartridge holder 28 has its corner parts at four locations each positioned in a state of being positioned inside the L-shaped ridge part 103 on the bottom surface 101 of the accommodating part 10. In a state in which the cartridge holder 28 is positioned in this manner, the through window 280 of the cartridge holder 28 matches the through window 100 of the accommodating part 10. Also, of the outer peripheral side walls 282A and 282B of the cartridge holder 28, the outer peripheral side walls 282A along the second direction are in a state of being apart from the inner side surface 105 of the accommodating part 10.

Of the outer peripheral side walls 282A and 282B of the cartridge holder 28 in a rectangular frame shape, as in FIG. 4 and FIG. 5, the outer peripheral side walls 282A on both sides along the second direction are provided with slits 282S in a height direction to the bottom part 281. The slits 282S are equidistantly provided at six locations in the second direction. The outer peripheral side walls 282A are configured of side wall pieces 282P split by the slits 282S to the bottom part 281. The positions of the slits 282S and the side wall pieces 282P are different between the outer peripheral side walls 282A on both sides, in a staggered manner.

On the side wall pieces 282P staggered on the outer peripheral side walls 282A on both sides, formation ranges in the second direction overlap, and a plurality of facing locations occur, where they face each other in the first direction. At each facing location on the side wall pieces 282P on both sides, a partition 285 is provided so that positions in the second direction match each other. The partitions 285 are equidistantly provided at five locations in the second direction so as to equally split the inner space of the cartridge holder 28 into six spaces 288 (FIG. 5). Each space 288 obtained by splitting is a space for accommodating the culture cartridge 2. Each space 288 has a front shape substantially matching the front shape of the culture cartridge 2. Also, on the outer peripheral side walls 282A on both end sides of the each space 288 divided in six parts, the slits 282S are positioned, one at each location, in a staggered manner.

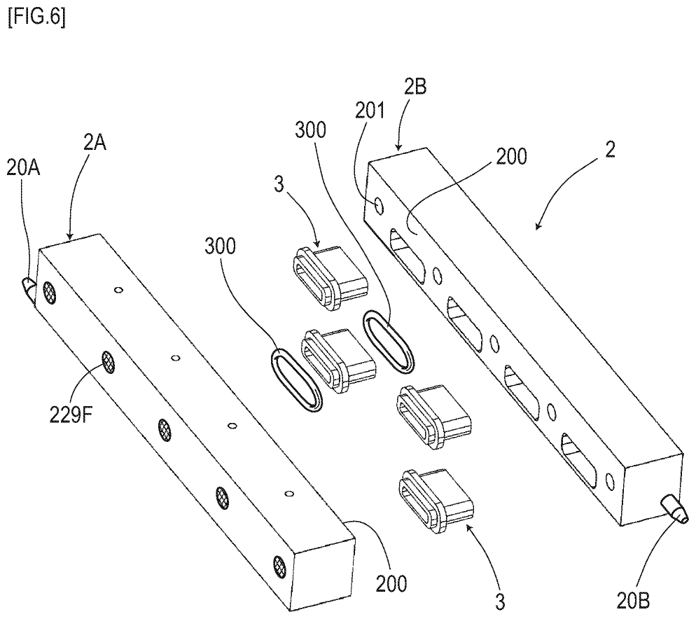

The culture cartridge 2 includes, as in FIG. 6, cell culture cartridges 3, which are one example of a sub-cartridge that is attachable and detachable. In the culture cartridge 2, the cell culture cartridge 3 is interposed between a holder 2A provided with a port 20A (one example of a connecting part) to which the silicone tube 178 on the upstream side forming the supply path is connected and a holder 2B provided with a port 20B (one example of the connecting part) to which the silicone tube 178 on the downstream side forming the collection path is connected. The holder 2A and the holder 2B each has an abutting surface 200, where a columnar magnet 201 is embedded, to face each other. The holder 2A and the holder 2B are bonded by being magnetically attracted each other by the magnetic force of the magnets 201.

The holders 2A and 2B and the cell culture cartridges 3 are all transparent products molded of acrylic resin. As a material of the holders 2A and 2B and the cell culture cartridges 3, polycarbonate, polystyrene, or the like may be adopted. The holders 2A and 2B and the cell culture cartridges 3 are preferably in a transparent or semitransparent state. In this case, a transparent or semitransparent side surface of the holders 2A and 2B and the cell culture cartridges 3 forms one example of the observation allowing part which allows cell observation, conveniently allowing the cell culture state to be observed from outside.

The cell culture cartridge 3 (FIG. 6 to FIG. 8) is a member having a flattened cylindrical shape and having a culture space 30 formed therein. At an opening end forming one end part of the cylindrical cell culture cartridge 3, a porous membrane 39 is provided, forming one example of a filter which can hold cells but allows the culture medium to pass therethrough. This porous membrane 39 is stuck at an end part of the cylindrical cell culture cartridge 3, and can also be peeled off for removal.

On the outer peripheral surface of the cell culture cartridge 3, a handguard-shaped flange 32 is provided partway in a cylindrical direction. On both sides of the flange 32, seal surfaces 32V orthogonal to the outer peripheral surface of the cell culture cartridge 3 are formed. In a state in which the cell culture cartridge 3 is assembled in the culture cartridge 2, O rings 300 are assembled as each being in a state of making contact with a relevant one of the seal surfaces 32V, achieving a fluid-tight state of the culture space 30.

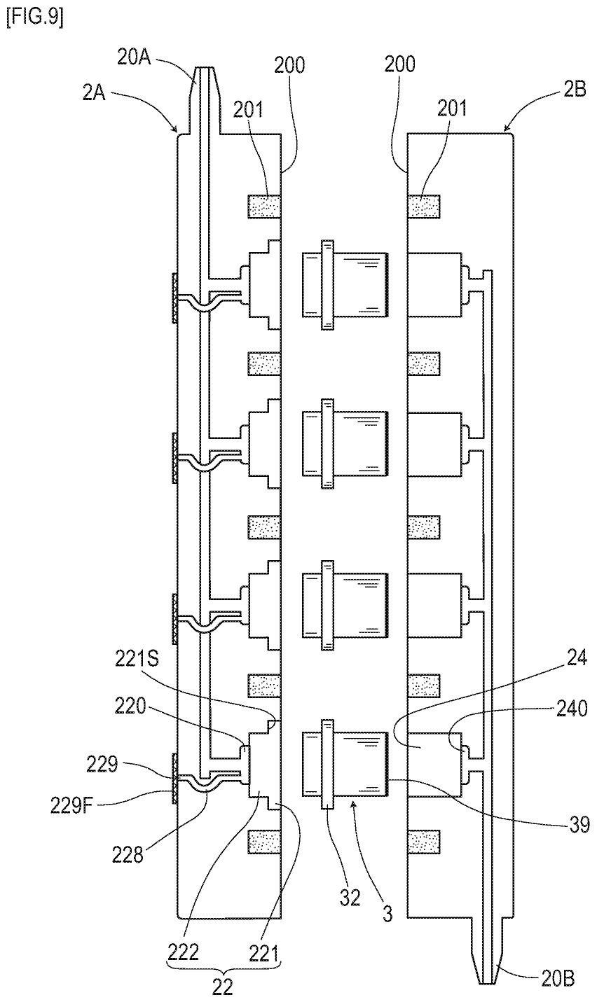

The holder 2A has, as in FIG. 9, the port 20A to which the silicone tube 178 (refer to FIG. 1 and the FIG. 2) forming the supply path is connected. The holder 2A has recessed parts 22 at four locations on the abutting surface 200, which is a surface facing the holder 2B. The recessed parts 22 are recessed parts each for accommodating the end part of the cell culture cartridge 3, which is an opening side opposite to the porous membrane 39. The recessed part 22 has a two-step structure with a shallow flange accommodating part 221 and a deep cylinder accommodating part 222 one size smaller than the flange accommodating part 221. The flange accommodating part 221 is a recessed part which accommodates the flange 32 of the cell culture cartridge 3. The cylinder accommodating part 222 is a recessed part which accommodates the end part of the cell culture cartridge 3. A bottom surface 221S of the flange accommodating part 221 functions as a seal surface for achieving fluid tightness by making contact with the O ring 300 assembled to the flange 32.

On the bottom surface of the cylinder accommodating part 222, a hole 220 communicating with the port 20A is open. Furthermore, a gas-draining path 228 communicates with this hole 220. This gas-draining path 228 communicates with outside via an air hole 229 provided by drilling the outer peripheral surface of the holder 2A. The air hole 229 is provided with a filter 229F forming one example of a member which allows gas to pass therethrough but does not allow the culture medium pass therethrough. According to the filter 229F with gas permeability and fluid tightness, when the culture medium is first supplied to the culture cartridge 2, it is possible to discharge gas remaining in the culture space 30 from the air hole 229 without leakage of the culture medium.

The holder 2B (FIG. 9) has the port 20B to which the silicone tube 178 (refer to FIG. 1 and FIG. 2) forming the collection path is connected. The holder 2B has recessed parts 24 individually corresponding to the recessed parts 22 at four locations on the holder 2A side. The recessed parts 24 are recessed parts each for accommodating the end part of the cell culture cartridge 3 on the side provided with the porous membrane 39. On the bottom surface of the recessed part 24, a hole 240 communicating with the port 20B is open.

The apparatus main body 1 is provided with, as in FIG. 10, air paths and culture medium paths for supplying the culture medium to the culture space 30. The culture medium paths include a supply path 10S which supplies the culture medium to the culture space 30, a collection path 10K which collects the culture medium passing through the culture space 30, and a recycle path 10R which returns the culture medium collected to the collection bottle 13B to the supply bottle 13A.

An air path 10T is a path through which the first pump 111 sends air to the supply bottle 13A. A suction side of the first pump 111 is connected to a path to the outside and is open to atmosphere. A discharge side of the first pump 111 is connected to a path via an air filter to the inside of the supply bottle 13A. In the apparatus main body 1, the internal pressure of the supply bottle 13A is increased by the discharge pressure of the first pump 111. By this internal pressure, the culture medium is pumped toward the culture space 30. The internal pressure of the supply bottle 13A is measured and controlled by the pressure sensor 130 (FIG. 3).

In the supply path 10S formed between the supply bottle 13A and the culture space 30, the seven-way coupling 17A on the upstream side is inserted. The supply path 10S is branched via the seven-way coupling 17A into six systems, which are connected to the ports 20A of six culture cartridges 2 held in parallel in the cartridge holder 28.

In the collection path 10K formed between the culture space 30 and the collection bottle 13B, the seven-way coupling 17B on the downstream side is inserted. The collection paths 10K of the six systems individually corresponding to the six culture cartridges 2 held in parallel in the cartridge holder 28 are collected via the seven-way coupling 17B into one system to reach the collection bottle 13B.

To the recycle path 10R from the collection bottle 13B to the supply bottle 13A, the second pump 112 is inserted. The second pump 112 suctions the culture medium accumulated in the collection bottle 13B and discharges the culture medium toward the supply bottle 13A. Unlike the configuration of the present embodiment, a configuration can be adopted in which the culture medium collected to the collection bottle 13B is not reused but discarded. By contrast, according to the configuration of the present embodiment in which the culture medium collected to the collection bottle 13B is returned to the supply bottle 13A for reuse, the culture work can be continued for a long time with respect to the amount of the culture medium. By replacing the culture medium when the culture medium is soiled, it is possible to efficiently use the culture medium.

Next, the procedure for culturing cells by using the above-configured culture apparatus 1A is described.

To culture cells by using the culture apparatus 1A, it is first required to seed cells in the cell culture cartridge 3. For example, by filling the culture space 30 with a gel such as collagen or agar for holding cells, cells can be seeded in the cell culture cartridge 3. The gel as a scaffold for holding cells is held in the culture space 30 by the porous membrane 39 forming the bottom surface of the culture space 30.

The culture cartridge 2 can simultaneously hold four cell culture cartridges 3 in parallel. To assemble the culture cartridge 2, four cell culture cartridges 3 with cells seeded and with the O rings 300 attached on both sides of the flange 32 are prepared. The four cell culture cartridges 3 are integrated so as to be interposed between the holders 2A and 2B to assemble the culture cartridge 2. The assembled state of the culture cartridge 2 can be held with high reliability by the magnetic force of the magnets 201 of the holders 2A and 2B.

In the culture cartridge 2, by the O ring 300 appropriately deformed between the bottom surface 221S of the flange accommodating part 221 of the holder 2A and the flange 32 of the cell culture cartridge 3, leakage of the culture medium supplied from the holder 2A to the culture space 30 is prevented. Also, by the O ring 300 appropriately deformed between the surrounding surface (abutting surface 200) of the recessed part 24 of the holder 2B and the flange 32 of the cell culture cartridge 3, the culture medium passing through the culture space 30 is supplied to the holder 2B without leakage.

Next, the culture cartridge 2 is set to the cartridge holder 28. As described above, the inner space of the frame-shaped cartridge holder 28 is split into the six equal spaces 288 by the partitions 285 provided inside. If the culture cartridge 2 is accommodated in this space 288, the dimension of the culture cartridge 2 in the height direction (direction in which the holder 2A and the holder 2B face each other) can be restricted by the partitions 285, and its assembled state can be held with even higher reliability.

In the cartridge holder 28, six culture cartridges 2 can be simultaneously set. After the cartridge holder 28 with six culture cartridges 2 set is prepared, the silicone tubes 178 penetrating through the inner side surface 105 of the accommodating part 10 are connected to the ports 20A and 20B protruding on both sides of the culture cartridges 2. To each port 20A of the holder 2A corresponding to the upstream side, six silicone tubes 178 exposed to the inner side surface 105 on the pump box 11 side are connected. To each port 20B of the holder 2B corresponding to the downstream side, six silicone tubes 178 exposed to the inner side surface 105 on the opposite side are connected.

The apparatus main body 1 with six culture cartridges 2 attached via the cartridge holder 28 in this manner is accommodated in the incubator not depicted, and the control cable 50 provided to extend from the external control unit 5 is connected to the apparatus main body 1. This allows the pumps 111 and 112 and so forth to be controlled by the control unit 5 and allows the culture medium to be supplied to the culture spaces 30 of the cell culture cartridges 3.

When supply of the culture medium is started, the control unit 5 first starts the operation of the first pump 111, which pumps air to the supply bottle 13A, controlling the first pump 111 so that the internal pressure of the supply bottle 13A is increased. When the internal pressure of the supply bottle 13A is increased, the culture medium accumulated in the supply bottle 13A is pushed and pumped toward the culture cartridges 2.

The culture medium pumped from the supply bottle 13A is branched into six systems by the seven-way coupling 17A, and flows into the holder 2A of each culture cartridge 2 via the silicone tubes 178. Here, the gas-draining path 228 communicates with the hole 220 for supplying the culture medium to the cylinder accommodating part 222 of the holder 2A. When the culture medium flows into the holder 2A, gas that is present in the cylinder accommodating part 222 of the holder 2A, the culture space 30 of the cell culture cartridge 3 and so forth is discharged outside via the gas-draining path 228, in accordance with the inflow of the culture medium. The air hole 229 bored in the outer peripheral surface of the holder 2A for discharging gas is provided with the filter 229F with gas permeability and fluid tightness. Thus, the culture medium is not leaked from this air hole 229.

After passing through the culture space 30, the culture medium supplied to the culture space 30 flows via the port 20B of the holder 2B into the seven-way coupling 17B on the downstream side. To this seven-way coupling 17B, six silicone tubes 178 connected to each port 20B of six culture cartridges 2 are connected. The culture medium flowing out from each culture cartridge 2 is collected by the seven-way coupling 17B to one system, and goes to the collection bottle 13B. Also, the culture medium collected in the collection bottle 13B is suctioned by the second pump 112 to be returned to the supply bottle 13A. The culture medium returned to the supply bottle 13A is supplied again to the culture cartridges 2 for use in cell culture. In the culture apparatus LA, this circulation of the culture medium allows cell culture to be continuously implemented. With the culture medium circulated, the amount of consumption of the culture medium, which is an expensive consumable article, can be reduced, and culture cost can be reduced.

Here, each culture cartridge 2 in the culture apparatus 1A of the present embodiment is held sideways along the horizontal direction by the cartridge holder 28, which forms a frame shape and has its upper surface side and lower surface side open. In the apparatus main body 1, this cartridge holder 28 is accommodated in the accommodating part 10 provided with the relatively large through window 100 on the bottom surface. Also, the holders 2A and 2B and the cell culture cartridges 3 forming the culture cartridge 2 are all molded of a transparent resin material.

In the culture apparatus LA, the cells seeded in the culture spaces 30 can be observed from above and below the culture cartridges 2 even during cell culturing. Also, the culture cartridges 2 can be removed as being in the cartridge holder 28. If the cartridge holder 28 is removed from the apparatus main body 1, observation of the cells being cultured is even easier. Furthermore, in the culture apparatus 1A of the present embodiment, the culture cartridges 2 can be removed one by one from the cartridge holder 28. Any of the six culture cartridges 2 can be removed for observation of the cells being cultured. Replacement of only any of the six culture cartridges 2 can be performed.

In the culture cartridge 2 in a state of being held in the cartridge holder 28 removed from the apparatus main body 1 or in a state of removed from the cartridge holder 28, the cells in the culture spaces 30 can be easily observed and, for example, placement on an observation table such as a microscope, is easy. In the present embodiment, the holders 2A and 2B and the cell culture cartridges 3 that are transparent are adopted, thereby allowing observation of the inside of the culture spaces 30. For the holders 2A and 2B and the cell culture cartridges 3, transparency is not an essential requirement. If these members have translucency, the cells in the culture spaces can be visually observed from outside.

Furthermore, in the culture apparatus 1A of the present embodiment, the culture cartridge 2 can be dissembled by separating the holders 2A and 2B, and the cell culture cartridges 3 can be removed. The holders 2A and 2B configuring the culture cartridge 2 are merely magnetically attracted, and thus can be easily separated to remove the cell culture cartridges 3. If the cell culture cartridge 3 is in a single state, the state of the cells in the culture space 30 can be even more easily observed, and placement to an observation table, such as a microscope, is even easier. While four cell culture cartridges 3 are held in the culture cartridge 2, replacement of only any part of the cell culture cartridges 3 can be performed.

In the apparatus main body 1, the culture space 30 during cell culturing can be observed from the upper surface side and the lower surface side. For example, if a light source such as a lighting fixture is arranged below the accommodating part 10, the cells can be observed by using transmitted light. The entire apparatus main body 1 can be set on an observation table such as a microscope. If the through window 100 of the accommodating part 10 is used, observation by a so-called biological microscope can be performed, in which an observation target is required to be positioned between an object lens and a reflecting mirror or the like. If the through window 100 of the accommodating part 10 is used, light reflected on the reflecting mirror or the like positioned below the culture space 30 where the cells are seeded can be caused to enter the object lens positioned above the culture space 30. In the culture apparatus 1A of the present embodiment, the observation allowing part which allows observation of cells from above or below in a direction orthogonal to the flowing direction of the culture medium is formed, and observation of a cross-sectional structure of the cells being cultured can be performed.

To extract the cells cultured in the culture space 30, for example, the porous membrane 39 at the opening end is peeled off, and then a member such as a plunger of a syringe is inserted into the culture space 30, thereby allowing cells to be pushed together with the gel as a scaffold or allowing the gel holding the cells to be suctioned from one opening end. In this manner, in the cell culture cartridge 3, the gel can be taken out along the cylinder direction of the culture space 30, and thus the cells can be taken out as the layer structure of the culture space 30 is kept. According to the culture apparatus LA, the cells can be extracted in a state extremely close to a culture state.

In the culture apparatus LA, by increasing the internal pressure of the supply bottle 13A, the culture medium can be pumped toward the culture space 30, and the hydraulic pressure of the culture medium can be caused to act in the cylinder direction of the culture space 30. If the hydraulic pressure of the culture medium is caused to act in the cylinder direction of the culture space in this manner, the hydraulic pressure of the culture medium can be caused to act on the cells, and the flow of the culture medium passing through the cells can be formed. The culture medium can be supplied with high reliability also to, for example, cells in an intermediate layer or lower layer of cells in a multilayered hierarchy, and necrosis of cells in an inner layer and so forth can be avoided. The culture apparatus 1A is an apparatus suitable also for culturing tissues with cells forming a multilayered structure.

On the other hand, in a conventional general culturing method in which, for example, cells are cultured as being placed in a stationary state on the bottom surface of a container such as a petri dish, a perfusion culturing method of continuously replacing only a supernatant fluid of the culture medium raises a high possibility that the culture medium cannot be sufficiently supplied to a deep part of tissues and cell aggregation. Therefore, in this culturing method, there is a high possibility that the period in which cells can be kept cannot be sufficiently ensured. Moreover, cells have a property of spreading along a bottom surface. Thus, in this culturing method, it is difficult to cause a flow of the culture medium in a direction of passing through the cells, and this method is unsuitable for culturing tissues with cells in a multilayered structure.

By contrast, in the culturing method by the culture apparatus 1A of the present embodiment, the porous membrane 39 is provided at the opening end of the cylindrical cell culture cartridge 3 on the downstream side and is taken as a bottom surface of the columnar culture space 30. In the culture apparatus 1A, the culture medium passes along the cylinder direction of the cylindrical cell culture cartridge 3, forming a flow of the culture medium passing through the cells held in the culture space 30. Therefore, in this culture apparatus 1A, the culture medium can be supplied to a deep part of the cells, and the cells can be stabilized and maintained over a long period of time.

For example, in metabolism of hepatic cells, there is a possibility that bile cannot be normally produced unless the culture medium flows through cells of three types, that is, stellate cells, sinusoidal endothelial cells, and then hepatic parenchymal cells, sequentially layered, in this order. If the flow of the direction of passing though the cells is achieved in this manner, culture under an environment closer to living body (in vivo) is possible. In the cylindrical cell culture cartridge 3, a multilayered structure of gels of a plurality of types with seeded cells of different types is preferably formed in the culture space 30.

In the culture apparatus LA, the hydraulic pressure of the culture medium can be caused to act on the cells seeded in the culture space 30. When pressure is applied to the cells, a perfusion can be caused to supply the culture medium to a tissue deep part of even thick vascularized tissues, and culture under conditions closer to living body can be achieved. If the acting pressure is controlled, the amount of fluid of the culture medium passing through vascularized tissues can be adjusted. This allows culture under optimum conditions.

As described above, the culture apparatus 1A is a useful apparatus which allows construction of hierarchical tissues by hepatic cells, construction of cancer cells with blood vessels, application to skin tissues and multipotential stem cells such as iPS cells and so forth.

In the culture apparatus LA, by adjusting the internal pressure of the supply bottle 13A, which is a supply source of the culture medium to the culture space 30, the hydraulic pressure and the flow rate of the culture medium passing through the cells can be adjusted. If the hydraulic pressure and the flow rate of the culture medium is finely adjusted, culture accuracy can be enhanced to improve reliability. In particular, in the culture apparatus 1A, a relatively large hydraulic pressure can be caused to act on the cells. Thus, the culture medium can be supplied also to, for example, tissues having a relatively large thickness on the order of 1 mm and having a large transmission resistance.

As the scaffold for seeding cells, collagen, agar, or the like may be used. When agar with a high transmission resistance is used as the scaffold, the culture medium is hard to be transmitted. On the other hand, according to the culture apparatus 1A of the present embodiment, by pressurizing the culture medium from outside, a relatively high hydraulic pressure can be achieved, and the culture medium can be transmitted even if the scaffold with a high transmission resistance such as agar is adopted.

In the culture apparatus 1A, by adjusting the internal pressure of the supply bottle 13A, the hydraulic pressure of the culture medium to be supplied to the culture space 30 can be controlled. For example, if the internal pressure of the supply bottle 13A is periodically increased and decreased, control of the hydraulic pressure simulating heartbeats or pulsation of blood pressure can be made. If control causing pressure fluctuations simulating pulsation of blood pressure is performed, cells under influences of pulsation of blood pressure can be cultured with high accuracy.

The collection bottle 13B or the supply bottle 13A may be provided with a pH sensor. For example, when a decrease in pH of the culture medium is detected by the pH sensor, circulation to the supply bottle 13A or supply to the culture cartridges 2 may be stopped. Also, a lamp may be provided which is lit up when a decrease in pH is detected to indicate the necessity of replacement of the culture medium.

In the present embodiment, as an observation allowing part for allowing observation of the cells in the culture space 30, the configuration for allowing visual or optical observation has been exemplarily described. Specifically, the opening of the accommodating part 10 with its upper surface open, the through window 100 on the bottom surface of the accommodating part 10, the through window 280 on the bottom surface of the frame-shaped cartridge holder 28, the side surfaces of the holders 2A and 2B and the cell culture cartridges 3 formed of transparent resin and so forth configure an observation allowing part. As for the observation allowing parts such as the opening of the accommodating part 10 with its upper surface open, the through window 100 on the bottom surface of the accommodating part 10, and the through window 280 on the bottom surface of the frame-shaped cartridge holder 28, a through structure is not essential, and the structure may be such that light is transmitted to the extent that observation can be optically performed. As for the observation allowing part formed by the side surfaces of the holders 2A and 2B formed of transparent resin, a window in a through structure may be used, as long as a fluid-tight state of the culture space 30 is ensured. In place of the present embodiment, if the cells are observed by, for example, an electron microscope or X-ray microscope, a portion configured so that transmittance of electron beams and X rays as a probe is high is preferably provided as the observation allowing part.

In the present embodiment, the configuration is exemplarily described in which, when the cartridge holder 28 with the culture cartridges 2 set therein is accommodated in the accommodating part 10, the silicone tubes 178 are manually connected to the ports 20A and 20B of the culture cartridges 2. In place of this, the configuration may be such that if the cartridge holder 28 is attached into the accommodating part 10, the port 20A is automatically connected to the supply path 10S and the port 20B is automatically connected to the collection path 10K.

In the foregoing, specific examples of the present invention are described in detail as in the embodiment, these specific examples merely disclose examples of technology included in the claims. Needless to say, the claims should not be restrictively construed based on the configuration, numerical values and so forth of the specific examples. The claims include techniques acquired by variously modifying, changing, or combining as appropriate the above-described specific examples by using known techniques, knowledge of a person skilled in the art and so forth.

### REFERENCE SIGNS LIST

1 apparatus main body
1A culture apparatus
10 accommodating part
10S supply path
10K collection path
100 through window (observation allowing part)
11 pump box
111 first pump
112 second pump
13A supply bottle
13B collection bottle
2 culture cartridge
2A, 2B holder
20A, 20B port (connecting part)
228 gas-draining path
229F filter
28 cartridge holder
280 through window (observation allowing part)
3 cell culture cartridge (sub-cartridge)
30 culture space
39 porous membrane (filter)

1. A culture apparatus which supplies a culture medium, which is a cell culture solution, to culture cells, comprising:
a culture cartridge including a culture space where the cells can be seeded;
a supply path for supplying the culture medium to the culture space; and
a collection path for collecting the culture medium passing through the culture space, wherein
the culture cartridge is attachable and detachable, and includes a connecting part for connecting, when attached, the culture space to the supply path and the collection path.

2. The culture apparatus according to claim 1, wherein the culture cartridge includes a sub-cartridge which is attachable and detachable, and the culture space is provided to the sub-cartridge.

3. The culture apparatus according to claim 1, wherein the apparatus includes an observation allowing part which allows observation of the cells seeded in the culture space of the culture cartridge in an attached state.

4. The culture apparatus according to claim 3, wherein the observation allowing part is provided so as to allow the cells to be observed from at least a direction orthogonal to a direction in which the culture medium passes through the culture space.

5. The culture apparatus according to claim 1, wherein the culture space is a space forming a columnar shape, and the culture space has a filter removably disposed at one end in a column direction of the culture space, the filter capable of holding the cells but allowing the culture medium to pass therethrough.

6. The culture apparatus according to claim 1, wherein a gas-draining path for discharging gas communicates with the culture space and the gas-draining path is provided with a member which allows gas to pass therethrough but does not allow the culture medium to pass therethrough.

7. The culture apparatus according to claim 2, wherein the apparatus includes an observation allowing part which allows observation of the cells seeded in the culture space of the culture cartridge in an attached state.

8. The culture apparatus according to claim 7, wherein the observation allowing part is provided so as to allow the cells to be observed from at least a direction orthogonal to a direction in which the culture medium passes through the culture space.

9. The culture apparatus according to claim 2, wherein the culture space is a space forming a columnar shape, and the culture space has a filter removably disposed at one end in a column direction of the culture space, the filter capable of holding the cells but allowing the culture medium to pass therethrough.

10. The culture apparatus according to claim 3, wherein the culture space is a space forming a columnar shape, and the culture space has a filter removably disposed at one end in a column direction of the culture space, the filter capable of holding the cells but allowing the culture medium to pass therethrough.

11. The culture apparatus according to claim 7, wherein the culture space is a space forming a columnar shape, and the culture space has a filter removably disposed at one end in a column direction of the culture space, the filter capable of holding the cells but allowing the culture medium to pass therethrough.

12. The culture apparatus according to claim 4, wherein the culture space is a space forming a columnar shape, and the culture space has a filter removably disposed at one end in a column direction of the culture space, the filter capable of holding the cells but allowing the culture medium to pass therethrough.

13. The culture apparatus according to claim 2, wherein a gas-draining path for discharging gas communicates with the culture space and the gas-draining path is provided with a member which allows gas to pass therethrough but does not allow the culture medium to pass therethrough.

14. The culture apparatus according to claim 3, wherein a gas-draining path for discharging gas communicates with the culture space and the gas-draining path is provided with a member which allows gas to pass therethrough but does not allow the culture medium to pass therethrough.

15. The culture apparatus according to claim 7, wherein a gas-draining path for discharging gas communicates with the culture space and the gas-draining path is provided with a member which allows gas to pass therethrough but does not allow the culture medium to pass therethrough.

16. The culture apparatus according to claim 4, wherein a gas-draining path for discharging gas communicates with the culture space and the gas-draining path is provided with a member which allows gas to pass therethrough but does not allow the culture medium to pass therethrough.

17. The culture apparatus according to claim 5, wherein a gas-draining path for discharging gas communicates with the culture space and the gas-draining path is provided with a member which allows gas to pass therethrough but does not allow the culture medium to pass therethrough.

18. The culture apparatus according to claim 9, wherein a gas-draining path for discharging gas communicates with the culture space and the gas-draining path is provided with a member which allows gas to pass therethrough but does not allow the culture medium to pass therethrough.

19. The culture apparatus according to claim 10, wherein a gas-draining path for discharging gas communicates with the culture space and the gas-draining path is provided with a member which allows gas to pass therethrough but does not allow the culture medium to pass therethrough.

20. The culture apparatus according to claim 12, wherein a gas-draining path for discharging gas communicates with the culture space and the gas-draining path is provided with a member which allows gas to pass therethrough but does not allow the culture medium to pass therethrough.
